(19) United States
(12) Patent Application Publication
(10) Pub. No.: US 2023/0002914 A1
(43) Pub. Date: Jan. 5, 2023

(54) RENEWABLE METHANOL PRODUCTION MODULE

(71) Applicant: SOUTHERN GREEN GAS LIMITED, Melbourne, Victoria (AU)

(72) Inventor: Rohan GILLESPIE, Melbourne (AU)

(73) Assignee: SOUTHERN GREEN GAS LIMITED, Melbourne, Victoria (AU)

(21) Appl. No.: 17/780,494

(22) PCT Filed: Oct. 13, 2020
(86) PCT No.: PCT/AU2020/051097

§ 371 Date: May 26, 2022

(30) Foreign Application Priority Data
Nov. 28, 2019 (AU) 2019904496

(51) Int. Cl. C25B 1/04; C25B 15/08; C07C 29/151; C07C 29/152

(52) U.S. Cl. CPC C25B 1/04; C25B 15/081; C07C 29/1516; C07C 29/152

(57) ABSTRACT

The present invention is directed to a renewable methanol production system generally comprising: 1. a water capture module for directly capturing water from air to provide water in a liquid form; 2. an electrolysis module for electrolysis of the liquid water to produce hydrogen; 3. an exothermic reactor for reacting the hydrogen from the electrolysis module with carbon dioxide to produce renewable methanol.

### TECHNICAL FIELD

The present invention relates broadly to a method of producing renewable methanol, and a renewable methanol production system. The invention further relates broadly to a renewable methanol production module and relates particularly, although not exclusively, to a plurality of co-located production modules together forming a renewable methanol production plant.

### BACKGROUND

It is known to react carbon dioxide with hydrogen at the required stoichiometric mixture to produce methanol. The carbon dioxide may be recovered from flue gas using known Carbon Capture and Storage (CCS) technology. The hydrogen may be obtained by the electrolysis of water with the electrolyser being powered by a renewable energy source, such as solar-derived power. This and other known technologies in this field suffer from at least the following problems:
i) the electrolyser requires a readily-available source of liquid water for electrolysis in the production of hydrogen;
ii) the production of hydrogen and/or carbon dioxide typically requires power derived from non-renewable sources and as such the methanol is not deemed renewable.

### SUMMARY OF INVENTION

According to a first aspect of the present invention there is provided a renewable methanol production module comprising:

a water capture generator designed for directly capturing water from atmosphere to provide water in a liquid form;

an electrolyser operatively coupled to the water capture generator for receiving the liquid water, the electrolyser being effective in electrolysis of the liquid water to produce hydrogen;

a reactor operatively coupled to the electrolyser for receiving the hydrogen and reacting it with carbon dioxide to produce renewable methanol.

Preferably the water capture generator includes an adsorbent material designed to be exposed to atmosphere for directly adsorbing water from the atmosphere onto the adsorbent material. More preferably the water capture generator also includes heating means designed to absorb heat from a renewable energy source and transfer it to the adsorbent material to release the adsorbed water from the adsorbent material to provide the liquid water for the electrolyser.

Preferably the renewable methanol production module also comprises a renewable electricity generating assembly powered by a renewable energy source, said electricity generating assembly configured to provide electricity for powering the electrolyser in electrolysis of the atmospheric water in the production of hydrogen. More preferably the renewable electricity generating assembly includes a plurality of solar panels operatively coupled to the electrolyser for powering it. Still more preferably the solar panels are coupled to the electrolyser via an inverter. Alternatively the solar panels are directly coupled to the electrolyser.

Preferably the solar panels are in the form of solar photovoltaic (PV) panels arranged in an elongate bank of panels. Alternatively the solar panels are in the form of printed solar membranes. More preferably the bank of solar PV panels are located in two (2) rows on respective of opposing faces of a solar framework structure which is oriented in a generally magnetic North to South direction. Still more preferably the solar framework structure is in cross-section shaped in the form of an isosceles triangle having each of the two (2) rows of PV panels mounted to respective of leg-sides of the solar framework structure for increased solar exposure for said solar panels.

Preferably the renewable methanol production module also comprises a carbon dioxide extractor for extracting carbon dioxide from atmosphere. More preferably the carbon dioxide extractor is configured for directly capturing carbon dioxide from atmosphere using a metal-organic framework (MOF) or other adsorbent structure capable of directly adsorbing carbon dioxide from the atmosphere.

Preferably the reactor is an exothermic reactor for reacting hydrogen from the electrolyser with carbon dioxide from the carbon dioxide extractor to produce renewable methanol. More preferably the exothermic reactor is operatively coupled to a heat exchanger designed to exchange heat derived from the production of renewable methanol with the carbon dioxide extractor to heat the adsorbent structure of said extractor to release the adsorbed carbon dioxide from the adsorbent structure. Alternatively the carbon dioxide extractor is operatively coupled to the renewable electricity generating assembly or a solar hot water heater for heating of the adsorbent structure to release the adsorbed carbon dioxide. Still more preferably the heat exchanger is operatively coupled to the electrolyser wherein steam produced from the exothermic reactor exchanges heat with the carbon dioxide extractor to promote the release of the adsorbed carbon dioxide wherein said steam is condensed to provide liquid water to the electrolyser for the production of hydrogen.

Preferably the water capture generator includes at least one pair of solar water heater panels mounted to a water capture framework structure associated with the solar framework structure. More preferably the water capture framework structure is of substantially the same configuration and aligned with the solar framework structure for increased solar exposure of the pair of water heater panels which are located on respective opposing sides of the water capture framework structure.

Alternatively the water capture generator is configured for directly capturing water from atmosphere using a MOF or other adsorbent structure capable of directly adsorbing water from the atmosphere. In this variation the heat exchanger associated with the exothermic reactor is operatively coupled to the water capture generator to heat the adsorbent structure of said water generator to release the adsorbed water from the adsorbent material. In this embodiment the water capture generator is operatively coupled to the carbon dioxide extractor wherein dehumidified air from the water capture generator is received by the carbon dioxide extractor for extracting carbon dioxide from the dehumidified air. In this case the carbon dioxide extractor is operatively coupled to the heat exchanger associated with the exothermic reactor to heat the adsorbent structure of the carbon dioxide extractor to release the adsorbed carbon dioxide. Alternatively the adsorbed carbon dioxide is released from the adsorbent structure by heating it using electricity provided by the renewable electricity generating assembly or a solar hot water heater.

Preferably the renewable methanol production module includes an equipment platform at which at least the electrolyser, the reactor, the heat exchanger, and the carbon dioxide extractor are located. More preferably the equipment platform is located alongside the solar framework structure and the water capture framework structure.

Preferably the carbon dioxide extractor is operatively coupled to one or more batteries charged by the electricity derived from the plurality of solar panels. More preferably the carbon dioxide extractor includes pumps and/or fans powered by electricity supplied from said one or more batteries.

Preferably the renewable methanol production module is one of a plurality of said production modules. More preferably said production modules are co-located and together form a renewable methanol production plant.

According to a second aspect of the present invention there is provided a method of producing renewable methanol comprising the steps of:

directly capturing water from air to provide water in a liquid form;

producing hydrogen by electrolysis of the liquid water;

reacting the hydrogen with carbon dioxide to produce renewable methanol.

Preferably the step of directly capturing water involves exposing air to an adsorbent material to adsorb water from the air onto the adsorbent material. More preferably said step also involves i) releasing the adsorbed water from the adsorbent material by heating it, and ii) condensing the released water by cooling it to provide the liquid water. Even more preferably the adsorbed water is released from the adsorbent material using a) solar energy, and/or b) waste heat from the reaction between hydrogen and carbon dioxide to heat the adsorbent material. Alternatively the step of directly capturing water involves refrigeration of air to release water from the air to provide the liquid water.

Preferably the step of producing hydrogen involves: i) generating electricity via a renewable energy source, and ii) using the electricity to power the electrolysis of the liquid water for the production of hydrogen.

Preferably the step of reacting the hydrogen with carbon dioxide involves a preliminary step of either extracting carbon dioxide from air or obtaining carbon dioxide from a biogas reactor. More preferably the extraction of carbon dioxide from air involves directly capturing carbon dioxide from air using solar energy and/or waste heat from the reaction between hydrogen and carbon dioxide.

Preferably the method also comprises the step of recirculating liquid water produced from the reaction between hydrogen and carbon dioxide for electrolysis in the production of hydrogen. More preferably the recirculated liquid water is combined with the liquid water directly captured from air for electrolysis in the production of hydrogen.

According to a third aspect of the invention there is provided a renewable methanol production system comprising:

a water capture module for directly capturing water from air to provide water in a liquid form;

an electrolysis module for electrolysis of the liquid water to produce hydrogen;

an exothermic reactor for reacting the hydrogen with carbon dioxide to produce renewable methanol.

Preferably the water capture module includes an adsorbent unit including an adsorbent material designed to be exposed to air for adsorbing water from the air onto the adsorbent material. More preferably the water capture module also includes i) a heating unit designed to heat the adsorbent material to release the adsorbed water from the adsorbent material, ii) a condensing unit designed to condense the released water by cooling it to provide the liquid water. Even more preferably the heating unit includes a) a solar heating unit, and/or b) a heat recovery unit associated with the exothermic reactor for recovering waste heat from the exothermic reaction, being arranged for heating of the adsorbent material.

Preferably the electrolysis module includes a renewable electricity generating module powered by a renewable energy source, said electricity generating module configured to provide electricity for powering the electrolysis of the liquid water in the production of hydrogen.

Preferably the production system also comprises a carbon dioxide module for either extracting carbon dioxide from air or obtaining carbon dioxide from a biogas reactor. More preferably the carbon dioxide module includes a carbon dioxide capture module for directly capturing carbon dioxide from air.

Preferably the production system also comprises a water recirculation module arranged to recirculate liquid water produced from the exothermic reactor to the electrolysis module for the production of hydrogen.

### BRIEF DESCRIPTION OF DRAWINGS

In order to achieve a better understanding of the nature of the present invention a preferred embodiment of a renewable methanol production module as well as a method and system for the production of renewable methanol will now be described, by way of example only, with reference to the accompanying drawings in which:

FIG. 1 is an enlarged plan view of part of an embodiment of a renewable methanol production module according to one aspect of the invention;

FIG. 2 is a perspective view of the renewable methanol production module of the embodiment of FIG. 1 shown in its entirety;

FIG. 3 is a schematic of a process flow sheet for a method and system for the production of renewable methanol according to another aspect of the invention.

### DETAILED DESCRIPTION

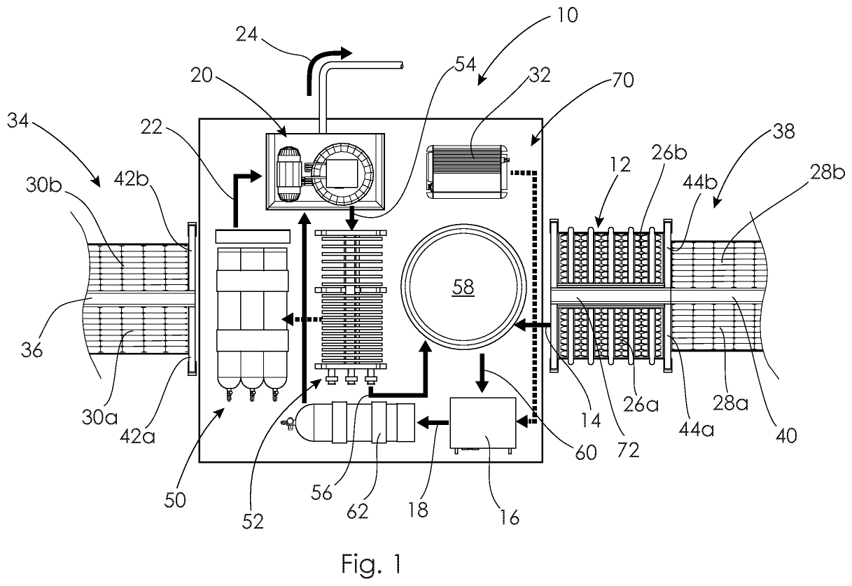

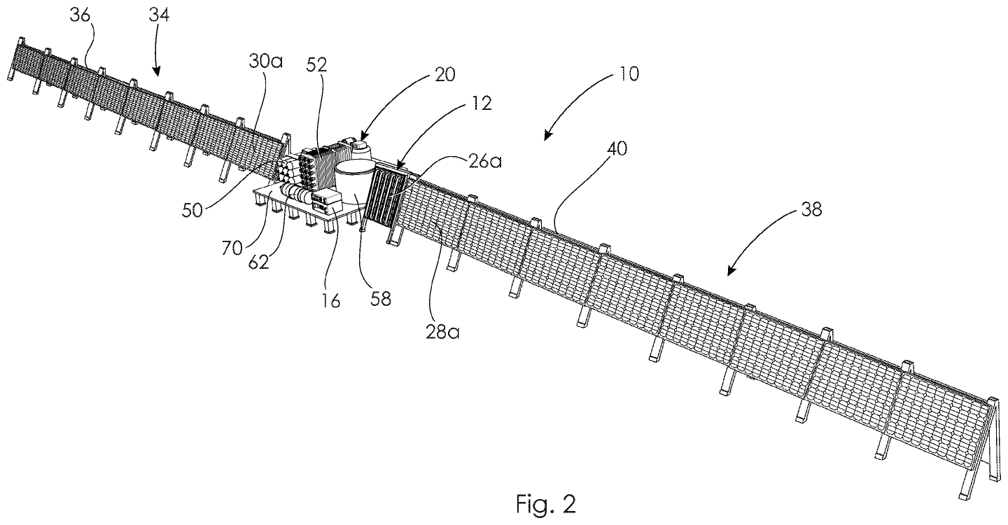

As seen in FIGS. 1 and 2 there is according to one aspect of the invention a renewable methanol production module 10 generally comprising:
1. a water capture generator 12 designed for directly capturing water from atmosphere to provide water in a liquid form at 14;
2. an electrolyser 16 operatively coupled to the water capture generator 12 for electrolysis of the liquid water to produce hydrogen at 18;
3. a reactor 20 operatively coupled to the electrolyser 16 for reacting the hydrogen with carbon dioxide at 22 to produce renewable methanol at 24.

In this embodiment the water capture generator 12 includes a pair of solar water heater panels 26a and 26b each including an adsorbent material (not shown) designed to be exposed to atmosphere for directly adsorbing water from the atmosphere on to the adsorbent material. Each of the water heater panels 26a/b of this embodiment also includes heating means (not shown) designed to absorb heat from a renewable energy source such as solar energy and transfer it to the adsorbent material to release the adsorbed water to provide water in a liquid form for the electrolyser 16. In this example the adsorbent material and solar heating means are together integrated within the water heater panels 26a/b.

In this embodiment the renewable methanol production module 10 also comprises a renewable electricity generating assembly (not designated) powered by a renewable energy source such as solar energy. The electricity generating assembly of this example includes a plurality of solar panels such as 28a/28b and 30a/30b operatively coupled to an inverter 32 for production of electricity for powering the electrolyser 16. In the absence of an inverter, the solar panels are directly coupled to the electrolyser 16. The solar panels such as 28a/b and 30a/b are in the form of solar photovoltaic (PV) panels arranged in an elongate bank of panels. In this case the solar PV panels are arranged in a first elongate bank of panels 34 in two rows such as 30a and 30b respectively on opposing faces of a first solar framework structure 36. The solar PV panels are also located in a second elongate bank of panels 38 in two rows of panels such as 28a and 28b respectively on opposing faces of a second solar framework structure 40. The first and second solar framework structures 36 and 40 are aligned with one another and oriented in a generally magnetic North to South direction. Each of the first and second solar framework structures 36/40 is in cross-section shaped in the form of an isosceles triangle having each of the two rows of PV panels such as 30a/b and 28a/b mounted to respective of leg-sides such as 42a/b and 44a/b of the first and second solar framework structures 36 and 40 respectively. It will be understood that this North to South orientation combined with the triangular solar framework structures 36 and 40 provides increased exposure of the solar PV panels 30a/b and 28a/b to sunlight.

In this embodiment the renewal methanol production module 10 also comprises a carbon dioxide extractor 50 for extracting carbon dioxide from atmosphere. The carbon dioxide extractor 50 directly captures carbon dioxide from atmosphere using a metal-organic framework (MOF) or other adsorbent structure (not shown). In this example the carbon dioxide extractor 50 is operatively coupled to a heat exchanger 52 to heat the adsorbent structure of the carbon dioxide extractor 50 to release the adsorbed carbon dioxide from the adsorbent structure. The released carbon dioxide at 22 is fed to the reactor 20 to react with the hydrogen in producing the renewable methanol at 24.

In this example the reactor 20 is an exothermic reactor which produces renewable methanol in an exothermic reaction. The production of methanol is promoted under appropriate reaction conditions and stoichiometry according to the equation:

CO₂+3H₂→CH₃OH+H₂O

This highly exothermic reaction is in this embodiment performed at a relatively high pressure of around 5 to 10 MPa and low temperature at around 250 to 300° C. in the presence of a CuO/ZnO/Al₂O₃ catalyst. It is to be understood that these reaction conditions and the catalyst may vary provided the production of methanol is favoured over other products derived from carbon dioxide and hydrogen as reactants.

The exothermic reactor 20 is operatively coupled to the heat exchanger 52 where steam at 54 from the reactor 20 exchanges heat with the carbon dioxide extractor 50 to release the adsorbed dioxide from the adsorbent structure associated with the carbon dioxide extractor 50. The steam on exchanging its heat at the heat exchanger 52 condenses to provide return liquid water at 56 to be circulated to the electrolyser 16 for the production of hydrogen.

In this embodiment the renewable methanol production module 10 comprises a water storage vessel 58 designed to store both the released water 14 from the water capture generator 12, and the return liquid water 56 from the heat exchanger 52. The water storage vessel 58 supplies the liquid water at 60 to the electrolyser 16 for the production of hydrogen. It is expected that the water capture generator 12 will supply liquid water to the storage vessel 58 predominantly during daylight hours when the adsorbed water from the adsorbent material of the water heater panels 26a/b is released on solar heating. The supply of the return liquid water 56 to the storage vessel 58 will occur during production of renewable methanol at the reactor 20 whilst steam is being condensed at the heat exchanger 52.

In this embodiment the renewable methanol production module 10 further comprises one or more hydrogen storage vessels 62 arranged to receive the hydrogen 18 produced from the electrolyser 16. The hydrogen storage vessel 62 is intended to provide an extended supply of hydrogen to the reactor 20 for continued operation without being limited to daylight hours during which water is predominantly released from the water capture generator 12. That is, the hydrogen storage vessel 62 provides an effective buffer in storing hydrogen for supply to the reactor 20. This hydrogen storage capability is consistent with operation of the electrolyser 16 during predominantly daylight hours when powered by the solar PV panels such as 28a/b and 30a/b and the associated inverter 32.

In this configuration the renewable methanol production module 10 includes an equipment platform 70 at which the electrolyser 16, the reactor 20, the inverter 32, the carbon dioxide extractor 50, the heat exchanger 52, the water storage vessel 58 and the hydrogen storage vessel 62 are located. The equipment platform 70 is in this embodiment located between the first and second solar framework structures 36 and 40. The pair of water heater panels 26a/b are mounted to a water capture framework structure 72 located adjacent the equipment platform 70. In this example the water capture framework structure 72 is of substantially the same configuration as and aligned with the second solar framework structure 40. It will be understood that this configuration provides the pair of water capture modules 26a/b with increased solar exposure in a similar manner to the solar PV panels such as 28a/b.

The carbon dioxide extractor 50 of this embodiment may be operatively coupled to one or more batteries (not shown) for extended operation without being limited to sunlight hours. In this configuration the inverter 32 is arranged to provide electricity for charging of the batteries. The electricity produced from the batteries may be used predominantly outside daylight hours for not only heating the MOF or other adsorbent structure of the carbon dioxide extractor 50 for releasing carbon dioxide but also to power pumps and/or fans (not shown) associated with the carbon dioxide extractor 50. The carbon dioxide extractor 50 is otherwise powered during daylight hours by the solar PV panels such as 28a/b and 30a/b via the associated inverter such as 32. This means the carbon dioxide extractor can potentially operate 24/7 in producing carbon dioxide for supply to the reactor 20 which likewise can operate around the clock. Although not illustrated, the carbon dioxide extractor may be operatively coupled to a solar hot water heater for heating of the adsorbent structure to release the adsorbed carbon dioxide.

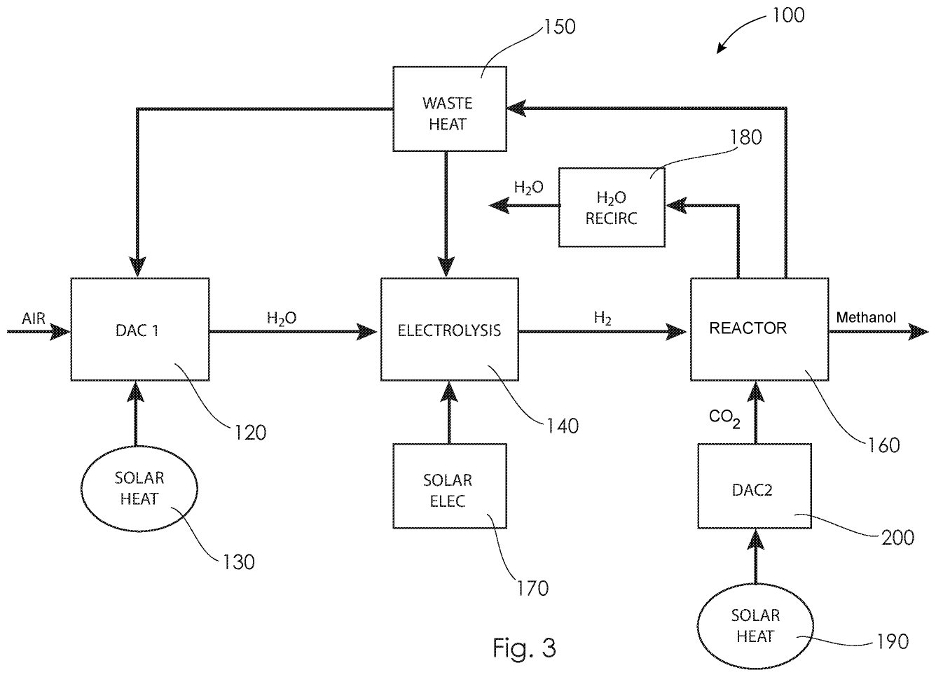

As seen in FIG. 3 there is according to another aspect of the invention a renewable methanol production system 100 generally comprising:
1. a water capture module 120 for directly capturing water from air to provide water in a liquid form;
2. an electrolysis module 140 for electrolysis of the liquid water to produce hydrogen;
3. an exothermic reactor 160 for reacting the hydrogen from the electrolysis module 140 with carbon dioxide to produce renewable methanol.

In this embodiment the water capture module 120 is in the form of a direct air capture module including a metal-organic framework (MOF) or other adsorbent designed to capture or adsorb water from the air. The MOF is the adsorbent material within an adsorbent unit of the water capture module 120. The water capture module 120 also includes i) a heating unit (not shown) designed to heat the MOF to release the adsorbed water, and ii) a condensing unit (not shown) designed to condense the water released from the MOF by cooling of the released water to provide the liquid water for the electrolysis module 140. In this example the heating unit includes a) a solar heating unit 130, and/or b) a heat recovery unit 150 associated with the exothermic reactor 160 for recovering waste heat from the associated exothermic reaction, in both cases the heating unit being arranged for heating of the MOF.

In this embodiment the electrolysis module 140 includes a renewable electricity generating module 170 powered by a renewable energy source, such as solar energy arranged to power an electricity generator (not shown) configured to provide electricity for powering the electrolysis module 140 for the production of hydrogen from the liquid water. It will be understood that the electrolysis module 140 may be powered by other renewable energy sources including but not limited to wind, wave, or tidal sources. The production system 100 of this embodiment also comprises a water recirculation module 180 arranged to recirculate liquid water produced from the exothermic reactor 160 to the electrolysis module 140 for the production of hydrogen.

In this embodiment the production system 100 also comprises a carbon dioxide module 200 for extracting carbon dioxide from air. The carbon dioxide module is based on MOF technology with the adsorbent material designed to adsorb carbon 200 dioxide from the air. The carbon dioxide capture module 200, in a similar manner to the water capture module 120, heats the adsorbent material such as the MOF via a solar heating unit 190. Alternatively the carbon dioxide may be obtained from a biogas reactor. In either case the carbon dioxide combines with hydrogen in the exothermic reactor 160 for the production of renewable methanol. In this example this reaction is an exothermic reaction where, under the influence of a suitable catalyst, carbon dioxide reacts with hydrogen to produce renewable synthetic methanol.

In a further aspect of the invention, in the context of the renewable methanol production system 100, there is a method of producing renewable methanol comprising the general steps of:
1. directly capturing water from air at the water capture module 120 to provide water in a liquid form;
2. producing hydrogen by electrolysis of liquid water at the electrolysis module 140;
3. reacting the hydrogen with carbon dioxide to produce renewable methanol at the exothermic reactor 160.

Now that a preferred embodiment of a renewable methanol production module and other aspects of the invention have been described it will be apparent to those skilled in the art that it has the following advantages:
1. the production module in production of renewable methanol is powered solely by renewable energy sources and in particular solar energy;
2. the renewable methanol production module and the other production systems are efficient in harnessing waste heat from the exothermic reactor to assist with direct capture of carbon dioxide from atmosphere;
3. the production module exploits the production of steam or liquid water in the reactor for return to the electrolyser in the production of hydrogen;
4. the production module in its preferred orientation of solar panels more effectively harnesses solar energy increasing utilisation of the electrolyser for extended production of hydrogen;
5. the production of renewable methanol is powered by or derived from renewable energy sources and in particular solar energy.

Those skilled in the art will appreciate that the invention as described herein is susceptible to variations and modifications other than those specifically described. For example, the specific number and configuration of the solar panels of the production module may vary from that described. The direct capture of water and/or carbon dioxide from atmosphere may be different to the MOF or other technologies of the preferred embodiment. For example, the direct capture of water from air may be effected by refrigeration using a reverse cycle air-conditioning system which is effective in releasing water from air in a liquid form. In this variation, the waste heat from the exothermic reaction may be harnessed in refrigeration of the air to release the liquid water. In another example, evacuated tubes may be used as an alternative heat source for releasing carbon dioxide from the adsorbent material of the carbon dioxide extractor. In the context of the production of renewable methanol, the liquid water from the reactor need not be recirculated to the electrolysis module. It is to be understood that references to solar panels extends to printed solar such as thin film PV.

It is to be understood that any acknowledgement of prior art in this specification is not to be taken as an admission that this prior art forms part of the common general knowledge as at the priority date of the claims.

All such variations and modifications are to be considered within the scope of the present invention the nature of which is to be determined from the foregoing description.

1. A renewable methanol production module comprising:
a water capture generator designed for directly capturing water from atmosphere to provide water in a liquid form;
an electrolyser operatively coupled to the water capture generator for receiving the liquid water, the electrolyser being effective in electrolysis of the liquid water to produce hydrogen; and
a reactor operatively coupled to the electrolyser for receiving the hydrogen and reacting it with carbon dioxide to produce renewable methanol.

2. A renewal methanol production module as claimed in claim 1 wherein the water capture generator includes an adsorbent material designed to be exposed to atmosphere for directly adsorbing water from the atmosphere onto the adsorbent material.

3. A renewal methanol production module as claimed in claim 2 wherein the water capture generator also includes heating means designed to absorb heat from a renewable energy source and transfer it to the adsorbent material to release the adsorbed water from the adsorbent material to provide the liquid water for the electrolyser.

4. A renewal methanol production module as claimed in claim 1, further comprising a renewable electricity generating assembly powered by a renewable energy source, said electricity generating assembly being configured to provide electricity for powering the electrolyser in electrolysis of the atmospheric water in the production of hydrogen.

5.-11. (canceled)

12. A renewal methanol production module as claimed in claim 1, further comprising a carbon dioxide extractor for extracting carbon dioxide from atmosphere.

13. A renewal methanol production module as claimed in claim 12 wherein the carbon dioxide extractor is configured for directly capturing carbon dioxide from atmosphere using a metal-organic framework (MOF) or other adsorbent structure capable of directly adsorbing carbon dioxide from the atmosphere.

14. A renewal methanol production module as claimed in claim 12, wherein the reactor is an exothermic reactor for reacting hydrogen from the electrolyser with carbon dioxide from the carbon dioxide extractor to produce renewable methanol.

15. A renewal methanol production module as claimed in claim 14 wherein the exothermic reactor is operatively coupled to a heat exchanger designed to exchange heat derived from the production of renewable methanol with the carbon dioxide extractor to heat the adsorbent structure of said extractor to release the adsorbed carbon dioxide from the adsorbent structure.

16. A renewal methanol production module as claimed in claim 14 wherein the carbon dioxide extractor is operatively coupled to the renewable electricity generating assembly or a solar hot water heater for heating of the adsorbent structure to release the adsorbed carbon dioxide.

17.-19. (canceled)

20. A renewal methanol production module as claimed in claim 15, wherein the water capture generator is configured for directly capturing water from atmosphere using a MOF or other adsorbent structure capable of directly adsorbing water from the atmosphere.

21. A renewal methanol production module as claimed in claim 20 wherein the heat exchanger associated with the exothermic reactor is operatively coupled to the water capture generator to heat the adsorbent structure of said water generator to release the adsorbed water from the adsorbent material.

22.-26. (canceled)

27. A renewal methanol production module as claimed in claim 12, wherein the carbon dioxide extractor is operatively coupled to one or more batteries charged by the electricity derived from the plurality of solar panels, the carbon dioxide extractor including pumps and/or fans powered by electricity supplied from said one or more batteries.

28. (canceled)

29. (canceled)

30. A renewal methanol production module as claimed in claim 27 wherein said production modules are co-located and together form a renewable methanol production plant.

31. A method of producing renewable methanol comprising the steps of:
directly capturing water from air to provide water in a liquid form;
producing hydrogen by electrolysis of the liquid water;
reacting the hydrogen with carbon dioxide to produce renewable methanol.

32. A method as claimed in claim 31 wherein the step of directly capturing water involves exposing air to an adsorbent material to adsorb water from the air onto the adsorbent material, said step also involving i) releasing the adsorbed water from the adsorbent material by heating it, and ii) condensing the released water by cooling it to provide the liquid water.

33.-35. (canceled)

36. A method as claimed in claim 31, wherein the step of producing hydrogen involves:
i) generating electricity via a renewable energy source, and
ii) using the electricity to power the electrolysis of the liquid water for the production of hydrogen.

37. A method as claimed in claim 31, wherein the step of reacting the hydrogen with carbon dioxide involves a preliminary step of either extracting carbon dioxide from air or obtaining carbon dioxide from a biogas reactor.

38. (canceled)

39. A method as claimed in claim 31, further comprising the step of recirculating liquid water produced from the reaction between hydrogen and carbon dioxide for electrolysis in the production of hydrogen.

40. (canceled)

41. A renewable methanol production system comprising:
a water capture module for directly capturing water from air to provide water in a liquid form;
an electrolysis module for electrolysis of the liquid water to produce hydrogen;
an exothermic reactor for reacting the hydrogen with carbon dioxide to produce renewable methanol.

42. A renewable methanol production system as claimed in claim 41, wherein the water capture module includes an adsorbent unit including an adsorbent material designed to be exposed to air for adsorbing water from the air onto the adsorbent material, the water capture module also including:
i) a heating unit designed to heat the adsorbent material to release the adsorbed water from the adsorbent material, and
ii) a condensing unit designed to condense the released water by cooling it to provide the liquid water.

43.-48. (canceled)
